(19) United States
(12) Patent Application Publication
(10) Pub. No.: US 2008/0051809 A1
(43) Pub. Date: Feb. 28, 2008

(54) Implant for Treating Rotator Cuff Injuiries

(75) Inventors: Michiel Verhelst, Izegem (BE); Emmanuel Audenaert, St-Michiels (BE)

Correspondence Address: JAMES C. WRAY, 1493 CHAIN BRIDGE ROAD, SUITE 300, MCLEAN, VA, 22101, US

(73) Assignee: Verde BVBA, Izegem (BE)

(21) Appl. No.: 11/659,873

(22) PCT Filed: Aug. 8, 2005
(86) PCT No.: PCT/IB05/02391

§ 371 Date: Feb. 9, 2007

(30) Foreign Application Priority Data
Aug. 9, 2004 (BE) 2004/0388

(51) Int. Cl. A61B 17/08; A61F 13/00; A61F 2/02

(52) U.S. Cl. 606/151; 602/44; 623/11.11

(57) ABSTRACT

The present invention concerns a gauze implant for treating cuff injuries. The said implant is composed of several, preferably eight, overlapping layers of gauze. The use of an implant composed of several layers, allows better stress distribution and ingrowth of tissue between the different layers.

The present invention concerns a gauze implant for treating rotator cuff injuries and a method for preparing such a gauze implant.

The shoulder is composed of three bony structures: the collarbone (clavicle), the upper arm bone (humerus) and the shoulder blade (scapula). The most important muscle for raising the arm is the deltoid muscle. With each contraction, however, this muscle exerts a destabilizing upward force on the shoulder joint. A strong group of muscles, called the rotator cuff, gives the shoulder its dynamic stability and neutralizes the destabilizing function of the deltoid muscle. The rotator cuff connects the humerus to the scapula and contributes to elevation and rotation of the arm. The four muscles forming the rotator cuff are the subscapularis, the supraspinatus, the infraspinatus and the teres minor. When these muscles contract, they pull on tendons, which moves the upper arm bone. However, the most important function of these muscles is to maintain the upper arm bone centred in the joint. If this function fails, the tissues between the upper arm bone and the bony roof of the shoulder are squashed causing pain and restriction of movement. When the joint is no longer centred, the otherwise evenly distributed stress on the articular cartilage surface changes and high peak stresses occur, eventually resulting in osteoarthritis of the joint. In a final stage, the upper arm bone collides with the bony roof of the shoulder, which will wear out.

Damage to the rotator cuff (e.g. a rupture) is caused by injuries such as shoulder joint dislocations, or by trying to catch a heavy object or lifting an object with an outstretched arm. Most injuries, however, are caused by age-related degenerative changes. Although rotator cuff ruptures are most common in middle-aged people, they may also occur at a younger age. When the tendons of the rotator cuff muscles are torn off, these muscles become non-functional and atrophied, which means fatty degeneration of muscle tissue. Even after repair of tendon continuity, these muscles remain weakened and are unable to optimally carry out their dynamically stabilizing function. Characteristic of a rotator cuff injury is a narrowed space between the upper arm bone and the bony roof of the shoulder.

If the rotator cuff has been torn off completely, operative repair of the tendon is necessary. This also applies to partial ruptures in case of severe pain.

The rotator cuff is repaired using an open or arthroscopic procedure. Usually the remnants of the tendon are removed first, after which the tendon is firmly sutured to the bone so as to obtain healing of the tendon to the bone.

Not all ruptures can be sutured; sometimes the rupture is too old and the muscle and tendon are retracted. In other—mostly elderly—patients the tissue has deteriorated and is no longer strong enough to be sutured. In these cases, synthetic implants are sometimes used to bridge the defect and close the gap. With such major defects, the synthetic implant will be attached to the remnants of the rotator cuff tendons, as well as to their original insertion site at the upper arm bone. As Lipmann Kessel wrote in 1982: “It is better not to try to restore; unless this may be done without any stress. Should there be any doubt, do not restore, and continue with decompression. In such cases we sometimes use carbon fibres or an insert piece to close the rupture”.

Carbon fibres for restoring and replacing ligaments used to be very promising and popular. However, carbon fibres have the disadvantage of being brittle and prone to crumbling. After some time, these tiny carbon fibre fragments may give rise to a reactive inflammation of the joint and to catabolic enzymatic reactions, as was the case in 80% of the patients. In all these cases the implanted material had to be operatively removed.

The new generation of polyester implants also have the above-mentioned disadvantages but to a far lesser extent and furthermore have good biocompatibility and good pulling properties. The existing implants, however, show minimal ingrowth of tendon and scar tissue. Because of the abrupt transition of the tendon to the implant, stress distribution is minimal, increasing the risk of re-rupture at this level.

The purpose of the invention is to manufacture an implant for restoring massive, irreparable rotator cuff ruptures, which does not have the above-mentioned disadvantages.

This purpose is achieved by manufacturing a gauze implant, which is composed of several, preferably eight, overlapping layers of gauze thickness of the layers is between 2 and 10 mm, more particularly between 2 and 7 mm, and preferably about 4 mm. The use of an implant composed of several layers, allows better stress distribution and ingrowth of tissue between the layers.

We indeed found that a narrowed space between the upper arm bone and the bony roof of the shoulder, as is the case in rotator cuff injuries, results in a dramatic loss of deltoid muscle power, interfering with full functional use of the arm. In healthy young people, the space between the upper arm bone and the bony roof of the shoulder is 10 mm.

The gauze implant restores the space between the upper arm bone and the bony roof of the shoulder, and will thus also recentre the joint, thanks to its passive spacer function, which normalizes the distribution of stress during movement and prevents cartilage damage from progressing to osteoarthritis. Repair of rotator cuff ruptures by synthetic implants without restoring the space between the upper arm bone and the bony roof of the shoulder, leads to functionally inferior results.

The gauze, preferably a fibre gauze, is more particularly made of synthetic material, preferably polyester, polypropylene or polyethylene.

According to the invention, for at least one layer of gauze, the openings of the gauze relative to the total surface of the layer amount to at least 50%.

According to the invention, in selected cases, the said implant is composed of at least four layers of fibre gauze, more particularly eight layers.

According to the invention, the gauze implant comprises a body preferably composed of eight layers, which can be cut in a shape corresponding with the size of the rotator cuff injury and a strong one-layer foot for anchoring the synthetic implant to the bone, more particularly the upper arm bone. The thickness of this implant is between 2 and 10 mm, more particularly between 2 and 7 mm, and preferably about 4 mm.

This patent application also concerns a method for preparing a gauze implant for treating rotator cuff injuries, whereby a one-layer gauze implant is folded at least one time in such a way that an implant of several layers is obtained.

According to the invention, the implant is preferably folded three times so as to obtain an implant of eight layers of gauze.

Optimally, according to the invention, the said implant, after having been folded, has a thickness between 2 and 10 mm, more particularly between 2 and 7 mm, and preferably about 4 mm.

Using this method and such a gauze implant, rotator cuff injuries can be repaired relatively simply, reducing the stress at the site of rupture to normal values with minimal postoperative retraction, stiffness or risk of new ruptures.

In order to further clarify the properties of the gauze implant and the method for manufacturing this implant, and to point out its additional advantages and particulars, a more detailed description of the method applied will now be provided. Obviously, nothing in the following description may be interpreted as a restriction of the protection of this invention demanded in the claims.

In this description, by means of reference numbers, reference is made to the attached drawings in which:

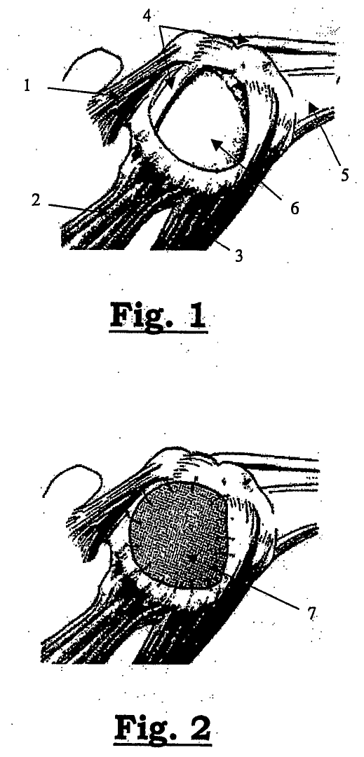

FIG. 1 is a representation of a ruptured rotator cuff with the subscapularis muscle (1), the supraspinatus muscle (2), the infraspinatus muscle (4), the biceps tendon (4) and the upper arm bone (humerus) (5);

FIG. 2 is a representation of a rotator cuff repaired by means of a gauze implant composed of several layers;

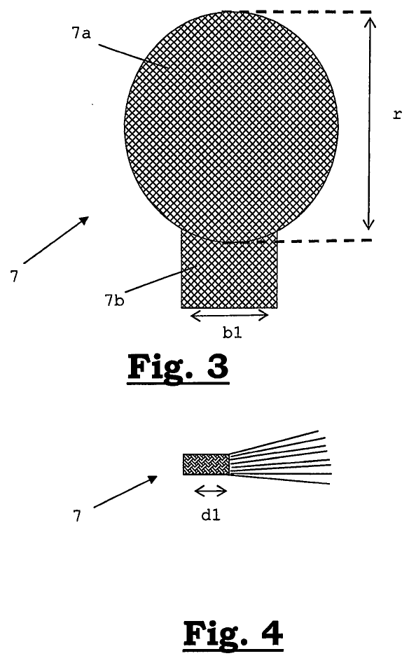

FIG. 3 is a top view of a gauze implant according to the invention;

FIG. 4 is a side view of a gauze implant according to the invention;

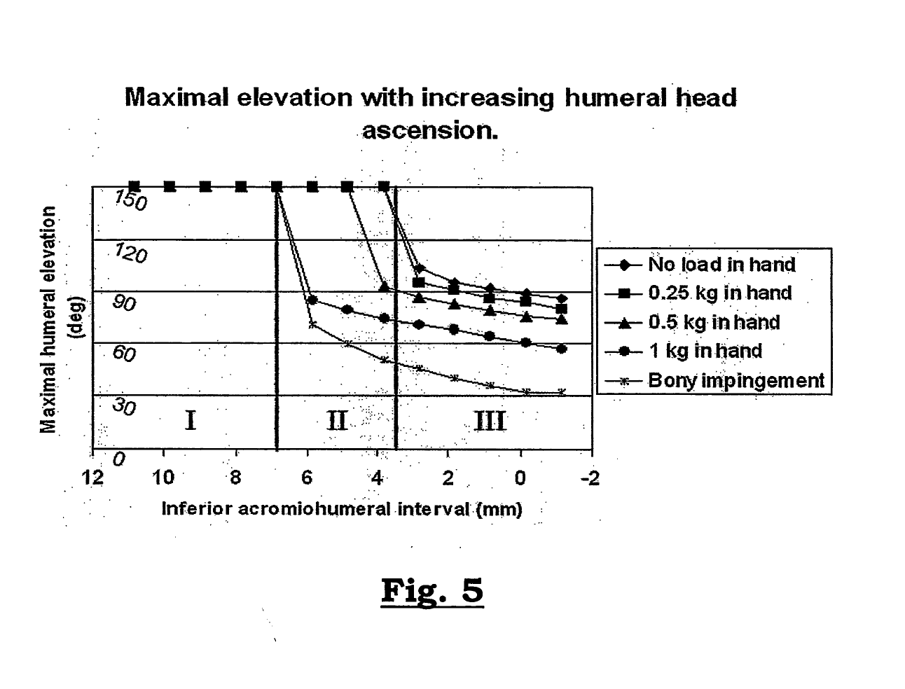

FIG. 5 represents the maximum elevation possible with a decreasing subacromial space.

From FIG. 5 it can be derived that narrowing of the space between the upper arm bone and the bony roof of the shoulder, as occurs in rotator cuff injuries, results in a dramatic loss of deltoid muscle power, interfering with full functional use of the arm.

In order to repair a rotator cuff defect, e.g. a rupture (6) of the tendon leaf of the rotator cuff (FIG. 1), a gauze implant (7) according to the invention can be used.

The said gauze implant (7) comprises a body (7a) composed of several, preferably eight, layers of fibre gauze. The thickness of the overlapping layers is between 2 and 10 mm, more particularly about 4 mm. Because the gauze implant will restore the space between the upper arm bone and the bony roof of the shoulder, it will also recentre the joint, thanks to its passive spacer function, which will normalize the stress distribution during movement and prevent cartilage damage from progressing to osteoarthritis.

On the other hand, the gauze implant may comprise a, strong one-layer foot (7b) for anchoring the synthetic implant to the bone, more particularly to the upper arm bone (5).

Preferably, the foot of the gauze implant has a width (b1) between 10 and 50 mm, more particularly 30 mm, and a depth (d1) between 5 and 20 mm, more particularly 10 mm. Preferably, the body (7a) oval-shaped, having a diameter (r) between 40 and 80 mm, more particularly 60 mm. The gauze implant may be cut in a shape corresponding with the size of the rotator cuff defect.

Repair of a rotator cuff defect, more particularly a rupture, by means of a gauze implant (7) according to the invention is performed under general anaesthesia. An incision is made in the shoulder and the ruptured tendonsheath of the rotator cuff is exposed. Remnants of the bursae are excised in order to avoid postoperative fluid formation. The remnants of the rotator cuff tendons are trimmed so that only strong tendon tissue remains. The gauze implant (7) is cut in a shape corresponding with the tendon defect and sewn onto the remnants of the rotator cuff tendons. The gauze implant is also secured to the bone at the site of the original rotator cuff tendons attachment. This will completely close the tendon defect.

1. Gauze implant (7) for treating rotator cuff injuries, characterized in that the said implant (7) is composed of several layers of gauze and the thickness of the overlapping layers is between 2 and 10 mm, more particularly about 4 mm.

2. Gauze implant (7) according to claim 1, characterized in that the said gauze (7) is a fibre gauze.

3. Gauze implant (7) according to claim 1, characterized in that the gauze is made of synthetic material, preferably polyester or polypropylene.

4. Gauze implant (7) according to claim 1, characterized in that, for at least one layer of gauze, the relation between the openings of the gauze and the total surface of the layer amounts to at least 50%.

5. Gauze implant (7) according to claim 1, characterized in that the said implant (7) is composed of at least four layers of fibre gauze, more particularly eight layers.

6. Gauze implant (7) according to claim 1, characterized in that the said implant (7) comprises a one-layer foot (7b) for anchoring the implant (7) to the bone.

7. Method for preparing a gauze implant for treating rotator cuff injuries, characterized in that the implant is folded at least one time in such a way that an implant (7) of several layers is obtained.

8. Method for preparing a gauze implant according to claim 7, characterized in that the implant is folded three times so as to obtain an implant (7) of eight layers of gauze.

9. Method for preparing a gauze implant according to claim 7, characterized in that the said implant (7), after having been folded, has a thickness between 2 and 10 mm, more particularly about 4 mm.
